(19) United States
(12) Patent Application Publication
(10) Pub. No.: US 2026/0120825 A1
(43) Pub. Date: Apr. 30, 2026

(54) SMALL LANGUAGE MODEL FOR MEDICAL INFORMATION EXTRACTION

(71) Applicant: Providence St. Joseph Health, Renton, WA (US)

(72) Inventors: M.V. Udai Shankar, Hyderabad (IN); Lalit Dhakar, Hyderabad (IN); Sri Pallavi M A, Hyderabad (IN); Kiran Pilli, Hyderabad (IN); Vankayala Achuta Murali Karthik, Guntur (IN); Preetha Kumar, Hyderabad (IN); Hari Atmakuri, Hyderabad (IN); Siddharth Yadav, Hyderabad (IN); Prashanth Pogu, Hyderabad (IN); Satyakumar Kolluri, Tirupati (IN)

(21) Appl. No.: 18/933,471

(22) Filed: Oct. 31, 2024

(51) Int. Cl. G16H 10/60

(52) U.S. Cl. CPC G16H 10/60

(57) ABSTRACT

A facility for training a small LLM model to extract medical information from patient notes is described. The facility receives an indication of one or more patient notes and masks at least a portion of the patient note. The facility trains a machine learning model with at least one embedding layer and a head layer based on the masked patient notes. The facility freezes at least one embedding layer of the machine learning model. The facility modifies the patient notes by changing at least one word in at least one of the patient notes to be a synonym. The facility modifies the machine learning model to replace the head layer with a second head layer. The facility trains the modified machine learning model based on the modified patient notes.

### BACKGROUND

Medical information is increasingly stored electronically, such as in the form of electronic health records that include notes regarding a patient that have been made by a health care provider. In some cases, patient notes are created by each health care provider that sees the patient, and such notes include information regarding the treatment, diagnoses, demeanor, concerns, other information relevant to the treatment or diagnosis of the patient, or some combination thereof. Health care providers may refer to patient notes for a particular patient to aid the diagnosis and treatment of the patient.

### BRIEF DESCRIPTION OF THE DRAWINGS

FIG. 1 is a block diagram showing some of the components typically incorporated in at least some of the computer systems and other devices on which the facility operates.

FIG. 2 is a flow diagram showing a process for training a tokenizer, used by the facility in some embodiments.

FIG. 3 is a flow diagram showing a process for generating synthetic patient note data, performed by the facility in some embodiments.

FIG. 4 is a sample prompt generation data table for generating synthetic patient note data, used by the facility in some embodiments.

FIG. 5 is a flow diagram showing a process for modifying patient notes to include noise and synonyms, performed by the facility in some embodiments.

FIG. 6 is a flow diagram showing a process for training a small LLM model, performed by the facility in some embodiments.

FIG. 7 is a block diagram of a sample small LLM model before embeddings are frozen, used by the facility in some embodiments.

FIG. 8 is a block diagram of a sample small LLM model after embeddings are frozen, used by the facility in some embodiments.

FIG. 9 is a flow diagram showing a process to re-train a small LLM model, performed by the facility in some embodiments.

FIG. 10 is a flow diagram showing a process for using a small LLM model, performed by the facility in some embodiments.

FIG. 11 is a flow diagram showing a process for changing the head of a small LLM model, performed by the facility in some embodiments.

FIG. 12 is a flow diagram showing a process for using a small LLM model with a medical question extraction head, performed by the facility in some embodiments.

### DETAILED DESCRIPTION

The inventor has recognized that it would be of great benefit to health care providers to be able to quickly understand patient notes gathered for a patient across a long period of time. However, because of the large volume of such patient notes over the course of a patient's care, it is not practical for an individual health care provider to adequately understand all of the patient notes for even a single patient, let alone all of the patients that may be cared for by the provider. Additionally, the inventor has also recognized that large language machine learning models (“LLMs”) may be used to process and summarize patient notes.

However, using publicly available LLMs or LLMs from third parties exposes patient health data to the public, which may be a violation of patient privacy. Additionally, such LLMs are cost prohibitive, because each token that may be included in even a single patient note, let alone patient notes accumulated over the patient's medical history, is factored into the cost for using such an LLM. A new prompt must also be generated for each use of these LLMs, thus a provider is unable to obtain a full idea of the patient's history. The inventor has further recognized that while a pretrained, and locally stored, LLM may be used to alleviate privacy concerns, this class of models require vast amounts of computing power and memory to run, which can be expensive to create, maintain, and use, and may require dedicated computing systems to create, maintain, and use such models. Furthermore, current LLMs are not trained to recognize specialized medical language that may be included in patient notes, and are instead trained on generalized terms that may not include any medical language. Thus, pretrained LLMs and public LLMs take a relatively long time to process and extract information from the patient notes in a way that would be useful to a medical provider.

As a result of these disadvantages, health care providers are currently unable to use artificial intelligence to adequately process and extract medical information from patient notes. Furthermore, conventional artificial intelligence models may miss key medical information helpful for providers in their diagnosis and treatment of patients.

In response to recognizing these disadvantages, the inventor has conceived and reduced to practice a software and/or hardware facility for medical information extraction (“the facility”). By training a customized LLM for one or more selected healthcare domains, the facility is able to provide health care providers with a model that can quickly process patient notes and extract medical information therefrom, and that is much smaller and more easy to operate than other conventional LLMs. Furthermore, because the customized LLM is smaller than conventional LLMs, it can run locally on a health care provider's computer, and thus prevent the exposure of patient data to the public.

The facility trains a customized LLM to extract information from patient notes in a selected healthcare or medical domain. In some embodiments, the size of the customized LLM is fewer than one-hundred-and-fifty megabytes. In some embodiments, the customized LLM has fewer than five million parameters. However, not all embodiments are so limited, and the model may be bigger in some embodiments or smaller in other embodiments. The customized LLM may operate without receiving a prompt and may receive as input only patient notes associated with a selected patient, from which medical information is to be extracted. The customized LLM outputs a dictionary of keys and values that is populated based on the extracted information. The values included in such a dictionary may be any data type, including text, integers, characters, Boolean, other dictionaries, other data types, or some combination thereof. The keys included in such a dictionary may relate to treatments, diagnoses, prognoses, tests, other types of medical information, or some combination thereof. The LLM may be trained to extract information associated with the keys of a dictionary.

The facility trains a tokenizer to convert one or more words or phrases included in patient notes into tokens. The LLM uses the output of the tokenizer to extract medical information from patient notes. The tokenizer is trained with sample patient notes, such as patient notes within a selected healthcare domain. A portion of the patient notes may be received from a repository that stores patient notes gathered from real patients and that have been released to the public, such as patient notes included in the MIMIC data set. A portion of the patient notes may be synthetic patient notes that are generated by the facility. The synthetic patient notes may be generated by generating a prompt based on a target dictionary associated with the selected healthcare domain and applying a machine learning model to the prompt to generate the patient notes. In some embodiments, the facility modifies patient notes to include noise before training the tokenizer. In some embodiments, the facility modifies a portion of the patient notes to replace one or more words or phrases in a portion of the patient notes with synonyms of the words or phrases.

The facility trains the customized LLM to extracting medical information with the tokenized patient notes to learn token and positional embeddings for the tokenized patient notes. In some embodiments, the facility masks at least a portion of the tokenized patient notes before training the customized LLM. In some embodiments, the masking is random, based on selected medical terms associated with a healthcare domain, or some combination thereof. The facility modifies the trained LLM by removing an output layer, also referred to as a “head layer,” of the trained LLM and replaces the output layer with a second output layer. The facility freezes the token and positional embedding layers of the LLM as part of modifying the trained LLM. The facility trains the modified LLM with tokenized patient notes to extract medical information from patient notes. In some embodiments, the modified LLM is trained to output a dictionary that includes the extracted medical information. In some embodiments, the trained LLM is a BART model.

The trained LLM may receive patient notes as input and extract medical information from the patient notes. The facility may train multiple head layers of the LLM, such as a head layer that outputs a dictionary that includes extracted medical information, a head layer that outputs a determination of whether patient notes indicate a selected medical condition, a head layer that outputs a determination of whether the patient notes indicate a specified medical condition, a head layer that outputs an answer to a specified health question, other head layers, or some combination thereof. In such embodiments, the facility may change the head layer of the LLM.

By performing in some or all of the ways described above, the facility is able to generate and use a “small” large language model for medical information extraction. Also, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, the small language model generated by the facility requires less memory, less processing power, and is able to operate more quickly due to its reduced size, when compared to conventional LLMs, because the small LLM is trained to recognize specific medical terms related to a selected medical domain. Also, the output of the small LLM can be changed to other types of output by changing the head layers of the small LLM. Thus, a single small LLM can perform a multitude of different functions that would require multiple larger LLMs to perform, without extensive retraining of the small LLM.

Further, for at least some of the domains and scenarios discussed herein, the processes described herein as being performed automatically by a computing system cannot practically be performed in the human mind, for reasons that include that the starting data, intermediate state(s), and ending data are too voluminous and/or poorly organized for human access and processing, and/or are a form not perceivable and/or expressible by the human mind; the involved data manipulation operations and/or subprocesses are too complex, and/or too different from typical human mental operations; required response times are too short to be satisfied by human performance; etc.

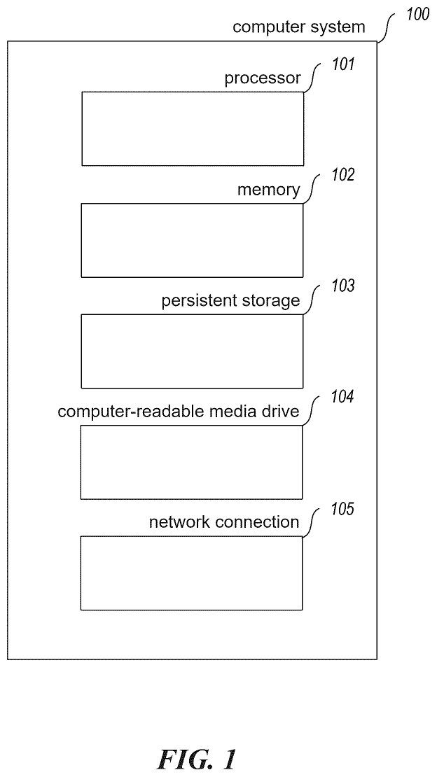

FIG. 1 is a block diagram showing some of the components typically incorporated in at least some of the computer systems and other devices on which the facility operates. In various embodiments, these computer systems and other devices 100 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 101 for executing computer programs and/or training or applying machine learning models, such as a CPU, GPU, TPU, NNP, FPGA, or ASIC; a computer memory 102—such as RAM, SDRAM, ROM, PROM, etc.—for storing programs and data while they are being used, including the facility and associated data, an operating system including a kernel, and device drivers; a persistent storage device 103, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. None of the components shown in FIG. 1 and discussed above constitutes a data signal per se. While computer systems configured as described above are typically used to support the operation of the facility, those skilled in the art will appreciate that the facility may be implemented using devices of various types and configurations, and having various components.

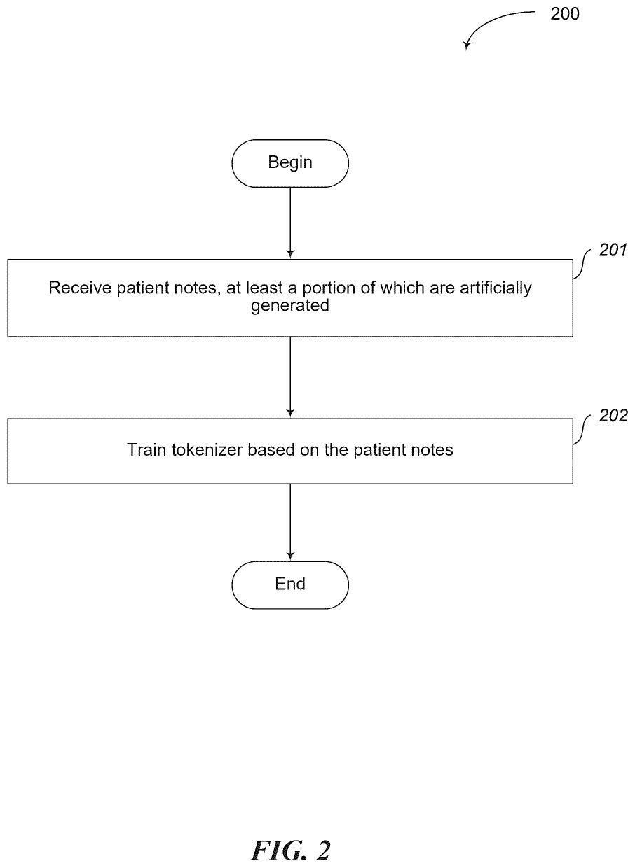

Those skilled in the art will appreciate that the acts shown in the flow diagrams of FIGS. 2, 3, 5, 6, 9, 10, 11, and 12 discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

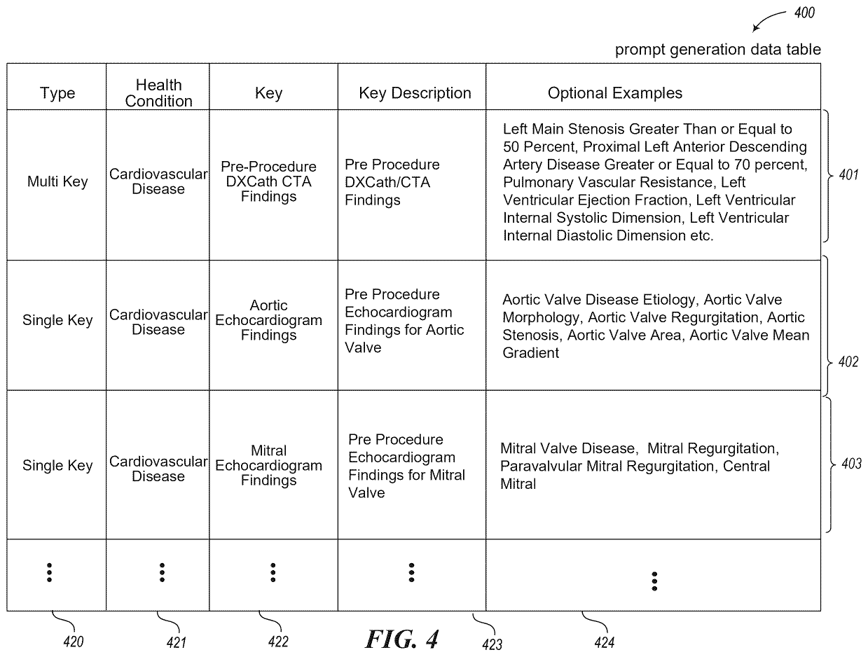

While the table diagram shown in FIG. 4 discussed below shows a table whose contents and organization are designed to make them more comprehensible by a human reader, those skilled in the art will appreciate that actual data structures used by the facility to store this information may differ from the table shown, in that they, for example, may be organized in a different manner; may contain more or less information than shown; may be compressed, encrypted, and/or indexed; may contain a much larger number of rows than shown, etc. Additionally, in some embodiments, rather than storing the data shown in the table diagrams in tables, the facility stores it in semi-structured or unstructured data stores, such as JSON objects.

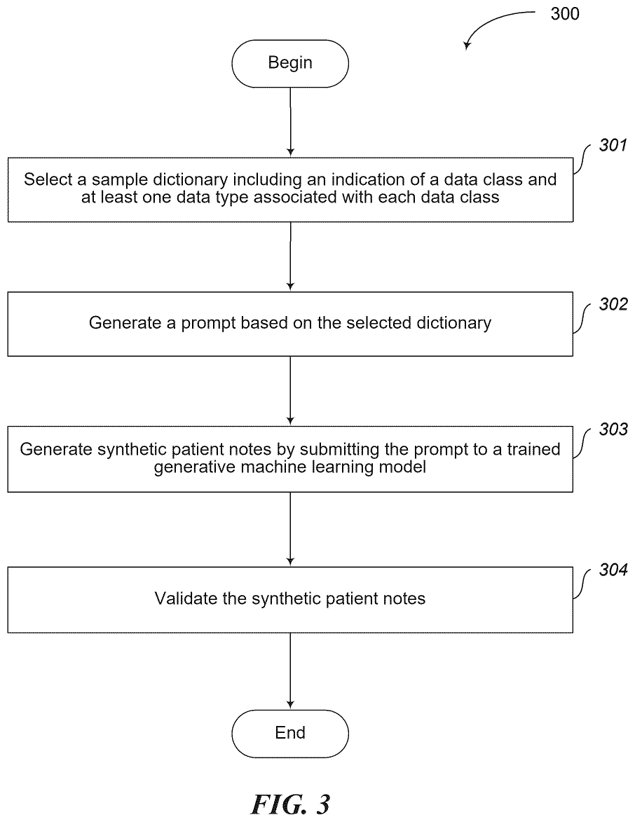

FIG. 2 is a flow diagram showing a process 200 for training a tokenizer, performed by the facility in some embodiments. First, at act 201, the facility receives a plurality of patient notes, at least a portion of which were artificially generated. In some embodiments, the facility receives at least a portion of the patient notes from a repository of patient notes, such as a repository storing: patient notes collected by an entity, patient notes included in a dataset such as the MIMIC dataset, patient notes generated by an entity, other sources of patient notes, or some combination thereof. In some embodiments, the facility uses the process 300 described below in connection with FIG. 3 to generate the artificially generated (or “synthetic”) patient notes.

FIG. 3 is a flow diagram showing a process 300 for generating synthetic patient note data, performed by the facility in some embodiments. First, at act 301, the facility selects a sample dictionary including an indication of a data class and at least one data type associated with each data class. In some embodiments, the facility selects the sample dictionary via a prompt generation data table for generating synthetic patient note data, such as the prompt generation data table 400, described below in connection with FIG. 4. In some embodiments, the facility selects one or more dictionaries randomly, based on a domain of medical knowledge, with other methods for selecting a dictionary, or some combination thereof.

FIG. 4 is a sample prompt generation data table 400 for generating synthetic patient note data, used by the facility in some embodiments. The prompt generation data table 400 includes a data type column 420, a health condition column 421, a data key column 422, a data key description column 423, and an optional example column 424. Although the prompt generation data table 400 includes data for generating synthetic notes regarding cardiovascular disease, embodiments are not so limited, and the prompt generation data table 400 may include data for generating synthetic notes regarding any health condition, treatment, patient behavior, other aspects of the care of a patient, or some combination thereof. Each row of the prompt generation data table 400 represents a definition of a dictionary that includes data associated with a domain of medical knowledge.

The data type column 420 includes an indication of whether the data included in a dictionary has a single key or multiple keys. In some embodiments, a single key data type indicates that the data included in the dictionary does not include another dictionary. In some embodiments, a multi key data type indicates that the data included in the dictionary includes another dictionary, thus a key included in the dictionary may be associated with another dictionary that includes its own keys and values. For example, the dictionary represented by row 401 is a multi key dictionary. Thus, the dictionary represented by row 401 includes a dictionary as one of the values, and a key, indicated by the key column 422, of the dictionary represented by row 401 has a value that corresponds to another dictionary included in the prompt generation data table 400. Continuing the example, the dictionaries represented by rows 402 and 403 are single key dictionaries, thus while the dictionaries represented by rows 402 and 403 may, in some embodiments, have multiple keys indicated by the key column 422, none of those keys are associated with another dictionary that includes its own keys and values.

The health condition column 421 includes an indication of a health condition for which the information included in the dictionary is related. For example, rows 401-403 indicate definitions of dictionaries that are related to cardiovascular disease. Although rows 401-403 indicate dictionaries associated with cardiovascular disease, embodiments are not so limited, and the prompt generation data table 400 may include dictionaries related to any number of different diseases, treatments, conditions, etc. In some embodiments, the health condition column 421 indicates a domain of medical knowledge, such as a certain health condition, treatment type, other domains of medical knowledge, or some combination thereof. For example, the health condition column 421 may indicate cardiovascular disease, cancer, chemotherapy, pediatric care, vaccinations, transplants, experimental research, or any other domain of medical knowledge.

The data key column 422 includes data indicating an identifier for one or more keys included in the dictionary. The data key description column 423 includes data indicating a description of the one or more keys indicated in the data key column 422. For example, the dictionary represented by row 401 has a key called “Pre-Procedure DXCath CTA Findings,” which represent the findings from a diagnostic catheterization procedure. As another example, the dictionary represented by row 403 has a key called “Mitral Echocardiogram Findings,” which represent the findings from a pre-procedure echocardiogram for the mitral valve.

The optional example column 424 includes data indicating one or more examples of the data that may be associated with a data key in the dictionary, and which may be included in a patient note generated based on the dictionary definition. For example, row 401 indicates that a patient note generated from the dictionary definition should have notes for diagnostic catheterization findings similar to “Left Main Stenosis Greater Than or Equal to 50 Percent, Proximal Left Anterior Descending Artery Disease Greater or Equal to 70 percent, Pulmonary Vascular Resistance, Left Ventricular Ejection Fraction, Left Ventricular Internal Systolic Dimension, Left Ventricular Internal Diastolic Dimension etc.” As another example, row 402 indicates that a patient note generated from the dictionary definition should have notes for pre-procedure aortic echocardiogram findings similar to “Aortic Valve Disease Etiology, Aortic Valve Morphology, Aortic Valve Regurgitation, Aortic Stenosis, Aortic Valve Area, Aortic Valve Mean Gradient.”

Returning to FIG. 3, at act 302, the facility generates a prompt based on the selected dictionary. In some embodiments, the facility generates prompt by applying one or more aspects of the selected dictionary to a template. Table 1 is an example of a prompt generated by the facility as part of performing act 302. Although the prompt indicated in Table 1 below specifies the creation of patient notes with noise (see Table 1 below, stating at its beginning “Create a noisy synthetic patient notes . . . ”), embodiments are not so limited, and the prompt may indicate that the synthetic patient notes are not to include noise, are to include synonyms of selected medical terms, other configurations of synthetic patient notes, or some combination thereof.

TABLE 1
Example Prompt

Create a noisy synthetic patient notes as recorded by a physician for a
cardiovascular disease patient with information of Pre Procedure DXCath/CTA
Findings: Like Left Main Stenosis Greater Than or Equal to 50 Percent, Proximal
Left Anterior Descending Artery Disease Greater or Equal to 70 percent,
Pulmonary Vascular Resistance, Left Ventricular Ejection Fraction, Left
Ventricular Internal Systolic Dimension, Left Ventricular Internal Diastolic
Dimension etc., Pre Procedure Echocardiogram Findings for Aortic Valve: Like
Aortic Valve Disease Etiology, Aortic Valve Morphology, Aortic Valve
Regurgitation, Aortic Stenosis, Aortic Valve Area, Aortic Valve Mean Gradient,
Pre Procedure Echocardiogram Findings for Mitral Valve: Like Mitral Valve
Disease, Mitral Regurgitation, Paravalvular Mitral Regurgitation, Central Mitral
Regurgitation, Mitral Stenosis, Mitral Valve Area, Mitral Valve Mean Gradient,
Mitral Valve Disease Etiology, Mitral Valve Annular Calcification, Pre Procedure
Echocardiogram Findings for Tricuspid Valve: Like Tricuspid Valve Disease
Etiology, Tricuspid Valve Regurgitation, Tricuspid Valve Diastolic Gradient,
Tricuspid Valve Annulus Size, Pre Procedure Echocardiogram Findings: Like
Leaflet Tethering, Mitral Valve Annular Calcification, End Diastolic Mid Right
Ventricle Diameter, End Diastolic Basal Right Ventricle Diameter, .
The patient notes should be atleast 300 words. It must be a free hand text
in single paragraph. The note text must contain characters only from [′a′, ′b′, ′c′, ′d′,
′e′, ′f′, ′g′, ′h′, ′i′, ′j′, ′k′, ′l′, ′m′, ′n′, ′o′, ′p′, ′q′, ′r′, ′s′, ′t′, ′u′, ′v′, ′w′, ′x′, ′y′, ′z′, ′A′, ′B′, ′C′,
′D′, ′E′, ′F, ′G′, ′H′, ′I′, ′J′, ′K′, ′L′, ′M′, ′N′, ′O′, ′P′, Q′, ′R′, ′S′, T, ′U′, ′V′, ′W′, ′X′, ′Y′,
′Z′, ′0′, ′1′, ′2′, ′3′, ′4′, ′5′, ′6′, ′7′, ′8′, ′9′, ′!′, ′″′, ′#′, ′$′, ′%′, ′&′, ″′″, ′(′, ′)′, ′*′, ′+′, ′,′ ′-′, ′.′,
′/′, ′:′, ′;′, ′<′, ′=′, ′>′, ′?′, ′@′, ′[′, ′\′, ′]′, ′\hat{ }′, ′_′, ′‘′, ′{′, ′|′, ′}′, ′~′, ′ ′].
The generated note should start with “patient notes:”
1. From the above patient notes extract metrics in the form of a dictionary
named D. If any of the metrics is not available, its value should be “None”.
2. The dictionary D should have the following keys:
Patient_Demographics, pre_procedure_dxcath_cta_findings,
aortic_echocardiogram_findings, mitral_echocardiogram_findings,
tricuspid_echocardiogram_findings, other_echocardiogram_findings, other_data.
3. The value corresponding to “Patient_Demographics” key should be a
dictionary with keys: [‘Last Name’, ‘First Name’, ‘Middle Name’, ‘Birth Date’,
‘SSN’, ‘SSN N/A’, ‘Sex’, ‘Patient Zip Code’, ‘Zip Code NA’, ‘Patient Race’,
‘Ethicity’] and the corresponding true values if available.
4. If the patient notes contains information about: Pre Procedure
DXCath/CTA Findings, the value corresponding to
‘pre_procedure_dxcath_cta_findings’: Like Left Main Stenosis Greater Than or
Equal to 50 Percent, Proximal Left Anterior Descending Artery Disease Greater
or Equal to 70 percent, Pulmonary Vascular Resistance, Left Ventricular Ejection
Fraction, Left Ventricular Internal Systolic Dimension, Left Ventricular Internal
Diastolic Dimension etc., should be a dictionary. Each parameter name should
be a key in this dictionary and the value should be a python 2-tuple consisting of
(True or False telling whether the parameter was drawn or not, parameter
value). ‘pre_procedure_dxcath_cta_findings’ should be an empty dictionary if the
patient notes does not contain this information.
5. If the patient notes contains information about: Pre Procedure
Echocardiogram Findings for Aortic Valve, the value corresponding to
‘aortic_echocardiogram_findings’: Like Aortic Valve Disease Etiology, Aortic
Valve Morphology, Aortic Valve Regurgitation, Aortic Stenosis, Aortic Valve
Area, Aortic Valve Mean Gradient, should be a 6-tuple consisting of (Aortic Valve
Disease Etiology: like Degenerative, Endocarditis, Rheumatic, Other; Aortic
Valve Morphology: like Bicuspid Aortic Valve, Tricuspid Valve, Other; Aortic
Valve Regurgitation: like None, Trace/Trivial, Mild, Moderate, Severe; True or
False whether Aortic Stenosis present or not; Aortic Valve Area; Aortic Valve
Mean Gradient). The value of ‘aortic_echocardiogram_findings’ should be None
if the patient notes does not contain this information.
6. If the patient notes contains information about: Pre Procedure
Echocardiogram Findings for Mitral Valve, the value corresponding to
‘mitral_echocardiogram_findings’: Like Mitral Valve Disease, Mitral
Regurgitation, Paravalvular Mitral Regurgitation, Central Mitral Regurgitation,
Mitral Stenosis, Mitral Valve Area, Mitral Valve Mean Gradient, Mitral Valve
Disease Etiology, Mitral Valve Annular Calcification, should be a 9-tuple
consisting of (True or False whether Mitral Valve Disease is there or not; Mitral
Regurgitation: Like None, Trace/Trivial, Mild, Moderate, Severe; Paravalvular
Mitral Regurgitation: Like None, Mild, Moderate, Severe; Central Mitral
Regurgitation: Like None, Mild, Moderate, Severe; True or False whether Mitral
Stenosis was performed; Mitral Valve Area; Mitral Valve Mean Gradient; Mitral
Valve Disease Etiology: Like Functional MR (Secondary), Degenerative MR
(Primary), Post Inflammatory, Other, None; True or False whether Mitral Valve
Annular Calcification is present or not). The value of
‘mitral_echocardiogram_findings’ should be None if the patient notes does not
contain this information.
7. If the patient notes contains information about: Pre Procedure
Echocardiogram Findings for Tricuspid Valve, the value corresponding to
‘tricuspid_echocardiogram_findings’: Like Tricuspid Valve Disease Etiology,
Tricuspid Valve Regurgitation, Tricuspid Valve Diastolic Gradient, Tricuspid
Valve Annulus Size, should be a 4-tuple consisting of (Tricuspid Valve Disease
Etiology: Like Primary, Secondary, Pacemaker Induced, Other; Tricuspid Valve
Regurgitation: Like None, Trace/Trivial, Mild, Moderate, Severe; Tricuspid Valve
Diastolic Gradient; Tricuspid Valve Annulus Size). The value of
‘tricuspid_echocardiogram_findings’ should be None if the patient notes does
not contain this information.
8. If the patient notes contains information about: Pre Procedure
Echocardiogram Findings, the value corresponding to
‘other_echocardiogram_findings’: Like Leaflet Tethering, Mitral Valve Annular
Calcification, End Diastolic Mid Right Ventricle Diameter, End Diastolic Basal
Right Ventricle Diameter, should be a 4-tuple consisting of (Leaflet Tethering:
Like None, Anterior Leaflet, Posterior Leaflet, Bileaflet; True or False whether
Mitral Valve Annular Calcification is there or not; End Diastolic Mid Right
Ventricle Diameter, End Diastolic Basal Right Ventricle Diameter). The value of
‘other_echocardiogram_findings’ should be None if the patient notes does not
contain this information.
9. If the patient notes contain extra information that is not covered under
any of the above keys, put these values corresponding to ‘other_data’. Each
information name should be a key in this dictionary and the value should be the
respective value. The value of ‘other_data’ should be None if the patient notes
does not contain this information.
The output must start with “ ” and should be followed by a JSON formatted text.
It should contain metrics present in the Patient Notes only. The output must
contain characters only from [′a′, ′b′, ′c′, ′d′, ′e′, ′f′, ′g′, ′h′, ′i′, ′j′, ′k′, ′l′, ′m′, ′n′, ′o′, ′p′, ′q′,
′r′, ′s′, ′t′, ′u′, ′v′, ′w′, ′x′, ′y′, ′z′, ′A′, ′B′, ′C′, ′D′, ′E′, ′F′, ′G′, ′H′, ′I′, ′J′, ′K′, ′L′, ′M′, ′N′,
′O′, ′P′, ′Q′, ′R′, ′S′, ′T′, ′U′, V′, ′W′, ′X′, ′Y′, ′Z′, ′0′, ′1′, 2′, ′3′, ′4′, ′5′, ′6′, ′7′, ′8′, ′9′, ′!′ ′″′,
′#′, ′$′, ′%′, ′&′, ″′″, ′(′, ′)′, ′*′, ′+′, ′,′ ′-′, ′.′, ′/′, ′:′, ′;′, ′<′, ′=′, ′>′, ′?′, ′@′, ′[′, ′\′, ′]′, ′\hat{ }′, ′_′,
′‘′, ′{{′, ′|′, ′}}′, ′~′, ′ ′]

At act 303, the facility generates synthetic patient notes by submitting the prompt to a trained generative machine learning model. In some embodiments, the trained generative machine learning model is a machine learning model trained to output text based on a prompt received by the machine learning model, such as, for example, GPT, Gemini, etc. Table 2 is an example of synthetic patient notes generated based on a prompt applied to a generative machine learning model. In some embodiments, the generated prompt includes instructions to the generative 5 machine learning model to generate a dictionary with a similar format to the selected dictionary based on a synthetic patient note generated by the generative machine learning model.

TABLE 2
Example Synthetic Patient Note.

Source, Target
“Patient notes: Patient presents today for Pre Procedure Echocardiogram
Findings: Leaflet Tethering, Mitral Valve Annular Calcification, End Diastolic Mid
Right Ventricle Diameter .5 cm, End Diastolic Basal Right Ventricle Diameter 5.9
cm, Pre Procedure Echocardiogram Findings for Tricuspid Valve: Tricuspid
Valve Disease Etiology: Functional, Tricuspid Valve Regurgitation: Trace,
Tricuspid Valve Diastolic Gradient: 1.0 mmHg, Tricuspid Valve Annulus Size: 2.8
cm, Pre Procedure Echocardiogram Findings for Mitral Valve: Mitral Valve
Disease: Rheumatic, Mitral Regurgitation: None, Paravalvular Mitral
Regurgitation: None, Central Mitral Regurgitation: Trace, Mitral Stenosis: Mild,
Mitral Valve Area: 2.3 cm2, Mitral Valve Mean Gradient: 7.1 mmHg, Mitral Valve
Disease Etiology: Rheumatic, Mitral Valve Annular Calcification: Mild, Pre
Procedure Echocardiogram Findings for Aortic Valve: Aortic Valve Disease
Etiology: Unknown, Aortic Valve Morphology: Normal, Aortic Valve
Regurgitation: None, Aortic Stenosis: Mild, Aortic Valve Area: 1.9 cm2, Aortic
Valve Mean Gradient: 11.1 mmHg.”, “{‘Patient_Demographics’: { },
‘other_echocardiogram_findings’: (‘None’, ‘True’, ‘0.5 cm’, ‘5.9 cm’),
‘tricuspid_echocardiogram_findings’: (‘Functional’, ‘Trace’, ‘1.0 mmHg’, ‘2.8 cm’),
‘mitral_echocardiogram_findings’: (False, ‘None’, ‘None’, ‘Trace’, True, ‘2.3 cm2’,
‘7.1 mmHg’, ‘Rheumatic’, ‘Mild’), ‘aortic_echocardiogram_findings’:
(‘UnkFalsewn’, ‘Normal’, ‘None’, True, ‘1.9 cm2’, ‘11.1 mmHg’), ‘other_data’: { }}”
“patient notes: Pre Procedure Echocardiogram Findings for Mitral Valve:
Like Mitral Valve Disease Grade 2+, Mitral Regurgitation Moderate, Paravalvular
Mitral Regurgitation None, Central Mitral Regurgitation None, Mitral Stenosis
None, Mitral Valve Area 1.4 Cm2, Mitral Valve Mean Gradient 4 Mm Hg, Mitral
Valve Disease Etiology Rheumatic, Mitral Valve Annular Calcification Mild; Pre
Procedure DXCath/CTA Findings: Like Left Main Stenosis Greater Than or
Equal to 50 Percent, Proximal Left Anterior Descending Artery Disease Greater
or Equal to 70 percent, Pulmonary Vascular Resistance 2.4 Wood Units, Left
Ventricular Ejection Fraction 55 Percent, Left Ventricular Internal Systolic
Dimension 3.2 Cm, Left Ventricular Internal Diastolic Dimension 4.5 Cm; Pre
Procedure Echocardiogram Findings for Tricuspid Valve: Like Tricuspid Valve
Disease Etiology Functional, Tricuspid Valve Regurgitation Moderate, Tricuspid
Valve Diastolic Gradient 4 Mm Hg, Tricuspid Valve Annulus Size 3.2 Cm; Pre
Procedure Echocardiogram Findings for Aortic Valve: Like Aortic Valve Disease
Etiology Calcific, Aortic Valve Morphology Bicommissural, Aortic Valve
Regurgitation None, Aortic Stenosis Mild, Aortic Valve Area 1.0 Cm2, Aortic
Valve Mean Gradient 12 Mm Hg.”, “{‘Patient_Demographics’: {‘Last Name’: None,
‘First Name’: None, ‘Middle Name’: None, ‘Birth Date’: None, ‘SSN’: None, ‘SSN
N/A’: None, ‘Sex’: None, ‘Patient Zip Code’: None, ‘Zip Code NA’: None, ‘Patient
Race’: None, ‘Ethicity’: None}, ‘mitral_echocardiogram_findings’: (True,
‘Moderate’, ‘None’, ‘None’, False, ‘1.4 Cm2’, ‘4 Mm Hg’, ‘Rheumatic’, False),
‘pre_procedure_dxcath_cta_findings’: {‘Left Main SteFalsesis Greater Than or
Equal to 50 Percent’: (False, None), ‘Proximal Left Anterior Descending Artery
Disease Greater or Equal to 70 percent’: (False, None), ‘Pulmonary Vascular
Resistance’: (False, None), ‘Left Ventricular Ejection Fraction’: (False, None),
‘Left Ventricular Internal Systolic Dimension’: (False, None), ‘Left Ventricular
Internal Diastolic Dimension’: (False, None)},
‘tricuspid_echocardiogram_findings’: (‘Functional’, ‘Moderate’, ‘4 Mm Hg’, ‘3.2
Cm’), ‘aortic_echocardiogram_findings’: (‘Calcific’, ‘Bicommissural’, ‘None’, False,
‘1.0 Cm2’, ‘12 Mm Hg’), ‘other_data’: { }}”
“patient notes: patient c/o chest pain, shortness of breath, and fatigue. pt has a
h/o HTN, HLD, DM, and CKD. ekG Shows sinus rhythm w/lvh and IBBB. Pre-
Procedure DXCath/CTA Findings: LMS >= 50%, Proximal LAD Disease >= 70%,
Pulmonary Vascular Resistance, Left Ventricular Ejection Fraction, Left
Ventricular Internal Systolic Dimension, Left Ventricular Internal Diastolic
Dimension. Pre-Procedure Echocardiogram Findings: Leaflet Tethering, Mitral
Valve Annular Calcification, End Diastolic Mid Right Ventricle Diameter, End
Diastolic Basal Right Ventricle Diameter. Pre-Procedure Echocardiogram
Findings for Mitral Valve: Mitral Valve Disease, Mitral Regurgitation, Paravalvular
Mitral Regurgitation, Central Mitral Regurgitation, Mitral Stenosis, Mitral Valve
Area, Mitral Valve Mean Gradient, Mitral Valve Disease Etiology, Mitral Valve
Annular Calcification. Pre-Procedure Echocardiogram Findings for Aortic Valve:
Aortic Valve Disease Etiology, Aortic Valve Morphology, Aortic Valve
Regurgitation, Aortic Stenosis, Aortic Valve Area, Aortic Valve Mean Gradient.
Pre-Procedure Echocardiogram Findings for Tricuspid Valve: Tricuspid Valve
Disease Etiology, Tricuspid Valve Morphology, Tricuspid Valve Regurgitation,
Tricuspid Valve Stenosis, Tricuspid Valve Area, Tricuspid Valve Mean Gradient.
I ordered a pre-procedure echocardiogram, which showed leaflet tethering and
mitral valve annular calcification. I also ordered a pre-procedure DXCath/CTA,
which showed LMS >= 50% and Proximal LAD Disease >= 70%. The patient will
need to undergo cardiac catheterization and possible
PCI.”, “{‘Patient_Demographics’: {‘Last Name’: None, ‘First Name’: None, ‘Middle
Name’: None, ‘Birth Date’: None, ‘SSN’: None, ‘SSN N/A’: None, ‘Sex’: None,
‘Patient Zip Code’: None, ‘Zip Code NA’: None, ‘Patient Race’: None, ‘Ethicity’:
None}, ‘other_echocardiogram_findings’: (None, True, None, None),
‘mitral_echocardiogram_findings’: (None, None, None, None, None, None,
None, None, True), ‘pre_procedure_dxcath_cta_findings’: {‘Left Main SteFalsesis
Greater Than or Equal to 50 Percent’: (True, ‘>=50%’), ‘Proximal Left Anterior
Descending Artery Disease Greater or Equal to 70 percent’: (True, ‘>=70%’),
‘Pulmonary Vascular Resistance’: (False, None), ‘Left Ventricular Ejection
Fraction’: (False, None), ‘Left Ventricular Internal Systolic Dimension’: (False,
None), ‘Left Ventricular Internal Diastolic Dimension’: (False, None)},
‘aortic_echocardiogram_findings’: (None, None, None, None, None, None),
‘other_data’: {‘Patient c/o’: [‘chest pain’, ‘shortness of breath’, ‘fatigue’], ‘h/o’:
[‘HTN’, ‘HLD’, ‘DM’, ‘CKD’], ‘ekG Shows’: [‘sinus rhythm w/lvh’, ‘IBBB’]}}”

In some embodiments, the generated synthetic patient note includes a dictionary with a format similar to the selected dictionary and that includes medical information included in the synthetic patient note. In some embodiments, the facility normalizes the data included in a dictionary included in a generated synthetic patient note, such that terms for the information included in the target dictionary are standardized. For example, the facility may change instances of “bp” to “blood pressure,” “hr” to “heart rate,” “patient demo” to “patient demographics,” “heart attack” to “cardiac arrest,” etc.

At act 304, the facility validates the synthetic patient notes. In some embodiments, the facility validates the synthetic patient notes by comparing the synthetic patient notes to the selected dictionary. In such embodiments, the facility may discard synthetic patient notes, or aspects of the synthetic patient notes, that are not able to be validated based on the selected dictionary. In some embodiments, the facility validates the synthetic patient notes by determining whether a dictionary has a multiple key value when it should instead have a single key value. In some embodiments, the facility validates the synthetic patient note by determining whether a dictionary has a single key value when it should instead have a multiple key value. In some embodiments, the facility validates the synthetic patient note based on a comparison of the values of one or more keys included in a dictionary of the synthetic patient note to the values of one or more keys included in the selected dictionary.

After act 304, the process 300 ends.

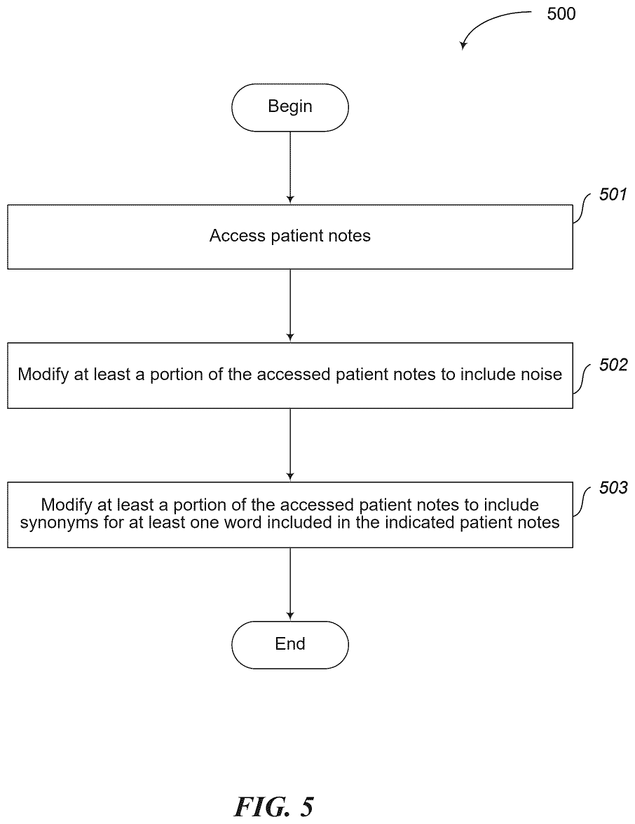

Returning to FIG. 2, at act 202, the facility trains a tokenizer to replace one or more words in patient notes with one or more tokens based on the received patient notes. In some embodiments, before training the tokenizer based on the received patient notes, the facility modifies the received patient notes to include noise, synonyms of one or more words included in the patient notes, or some combination thereof, such as by using the process 500 described below with respect to FIG. 5.

In some embodiments, the tokenizer is trained to normalize one or more words or phrases included in the patient notes. The facility may train the tokenizer to normalize one or more words or phrases included in the patient notes by using patient notes modified to include synonyms of one or more words included in the patient notes, by normalizing one or more words included in the received patient notes, or some combination thereof. For example, the tokenizer may be trained to recognize that the term “blood pressure” is a synonym of “bp,” and thus, replace the terms “blood pressure” and “bp” with the same token.

After act 202, the process 200 ends.

FIG. 5 is a flow diagram showing a process 500 for modifying patient notes to include noise and synonyms, performed by the facility in some embodiments. First, at act 501, the facility accesses one or more patient notes. In some embodiments, at least a portion of the accessed patient notes are synthetic patient notes. In some embodiments, the facility performs act 501 in a similar manner to act 201, described above in connection with FIG. 2. In some embodiments, the facility uses patient notes that have already been accessed, such as the patient notes described above in connection with the process 200.

At act 502, the facility modifies at least a portion of the accessed patient notes to include noise, such as random data, data unrelated to the medical domain for which a small LLM is being trained, etc. In some embodiments, the facility modifies the portion of the accessed patient notes by generating a prompt for a generative machine learning model to modify a patient note to include noise. In some embodiments, the facility identifies noise in one or more patient notes that were created by a healthcare provider for a patient, such as patient notes included in a repository that stores patient notes gathered from real patients and that have been released to the public. In such embodiments, the facility may use the identified noise to modify other patient notes to include noise. In some embodiments, the facility may modify one or more portions of patient notes to include noise based on a determination that the one or more portions include medical information associated with a particular medical domain. In some embodiments, modifying a patient note to include noise includes adding unnecessary information between patient notes, changing the order of one or more portions (such as, for example, changing the order of sentences) in the patient note, replacing words or phrases in the patient note with synonymous words or phrases, adding one or more spelling errors to one or more words or phrases in the patient note, adding one or more sentences at the beginning or end of the patient note that may not contain information related to the output, identifying a location where a particular word or phrase is found and removing a portion including the particular word or phrase, adding a portion of another patient note to the patient note, other methods of including noise, or some combination thereof.

At act 503, the facility modifies at least a portion of the accessed patient notes to include synonyms for at least one word included in the indicated patient notes. In some embodiments, the facility performs act 503 by, for at least one of the one or more patient notes, modifying at least one word included in the patient note to be a synonym of the at least one word.

After act 503, the process 500 ends.

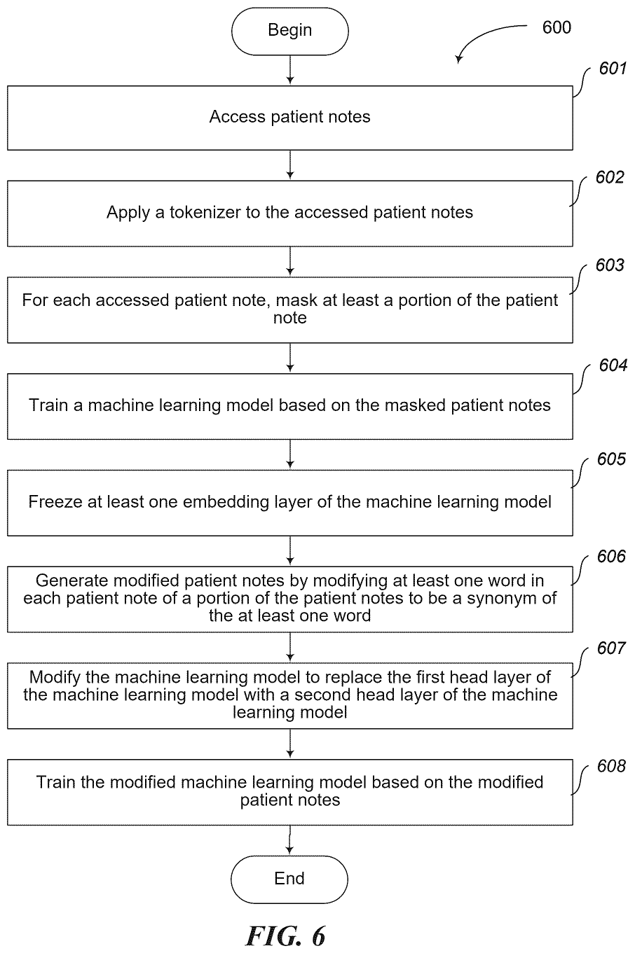

FIG. 6 is a flow diagram showing a process 600 for training a small LLM model, performed by the facility in some embodiments. First, at act 601, the facility accesses a plurality of patient notes. In some embodiments, the facility performs act 601 in a similar manner to act 501 described above with respect to FIG. 5. In some embodiments, the accessed notes are generated by the facility by using the process 500 described above with respect to FIG. 5.

At act 602, the facility applies a tokenizer to the accessed patient notes to obtain tokenized patient notes. In some embodiments, the tokenizer is a tokenizer generated by using the process 200 shown in FIG. 2 and described above.

At act 603, for each accessed patient note, the facility masks at least a portion of the patient note. In some embodiments, the facility masks one or more random words included in the patient note. In some embodiments, the facility masks one or more selected words included in the patient note. In such embodiments, the facility may select words to be masked that are related to the medical domain for which the small LLM model is to be trained.

At act 604, the facility trains a machine learning model, such as a small LLM model, based on the masked patient notes. In some embodiments, the facility trains the machine learning model to predict which tokens included in patient notes were masked. In some embodiments, the head layer of the machine learning model trained in act 604 is a head layer that outputs a prediction of which tokens in a set of patient notes were masked.

At act 605, the facility freezes at least one embedding layer of the trained machine learning model, such that subsequent training of the machine learning model does not alter the weights learned in the at least one embedding layer. In some embodiments, the facility freezes a token embedding layer and a positional embedding layer of the trained machine learning model.

At act 606, the facility generates modified patient notes by modifying at least one word in each patient note of a portion of the patient notes to be a synonym of the at least one word. In some embodiments, the facility performs act 606 in a similar manner to act 503, described above with respect to FIG. 5.

At act 607, the facility modifies the machine learning model to replace the first head layer of the machine learning model with a second head layer of the machine learning model. In some embodiments, the second head layer is a conditional generation head layer that outputs a dictionary that includes medical information extracted from one or more patient notes. In some embodiments, the second head layer is a head layer that outputs a determination of whether one or more patient notes indicate a specified medical condition. In some embodiments, the second head layer is a head layer that outputs a determination of whether one or more patient notes indicate a serious health condition. In some embodiments, the second head layer is a head layer that outputs an answer to a specified health question.

At act 608, the facility trains the modified machine learning model based on the modified patient notes. At act 608, because the at least one embedding layers are frozen, the at least one embedding layers are not changed when it is trained again. In some embodiments, the training data for the modified machine learning model includes one or more “target” dictionaries associated with the modified patient notes. In such embodiments, the data included in the target dictionaries may be “normalized,” such that terms for the information included in the target dictionary are standardized.

After act 608, the process 600 ends.

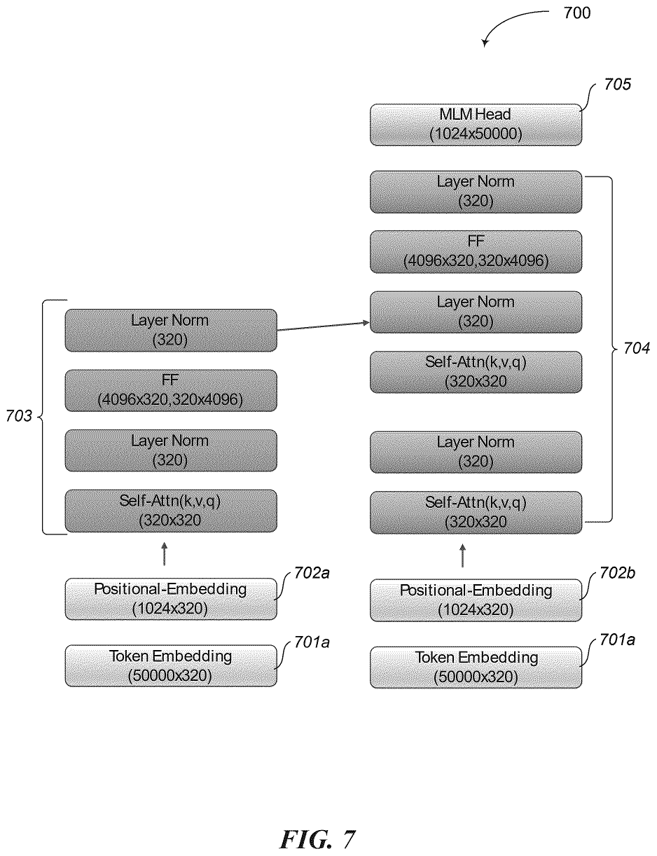

FIG. 7 is a block diagram of a sample small LLM model 700 before embeddings are frozen, used by the facility in some embodiments. The small LLM model 700 includes one or more token embedding blocks 701a and 701b (collectively “token embedding blocks 701” or individually as “token embedding block 701”), one or more positional embedding blocks 702a and 702b (collectively as “positional embedding blocks 702” or individually as “positional embedding block 702”), one or more encoder layers 703, one or more decoder layers 704, and an output layer (also referred to as a “head”) 705.

The token embedding blocks 701 are each embedding blocks that learn which terms included in patient notes have been tokenized. The positional embedding blocks 702 are each embedding blocks that learn the positions of terms included in patient notes that have been tokenized.

The encoder layer 703 includes one or more layer normalization blocks, one or more self-attenuation blocks, and one or more feed-forward (“FF”) blocks. The encoder layer 703 receives input from a positional embedding block, such as the positional embedding block 702a. The encoder layer 703 may transmit its output to one or more decoder layers, such as the decoder layer 704. In some embodiments, the small LLM model 700 includes multiple encoder layers 703. In an example embodiment, the small LLM model 700 includes 3 encoder layers.

The decoder layer 704 includes one or more self-attenuation blocks, one or more layer normalization blocks, and one or more FF blocks. The decoder layer 704 receives, as input, the output of one or more encoder layers, such as the encoder layer 703, and the output of a positional embedding block, such as the positional embedding block 702b. In some embodiments, the small LLM model 700 includes multiple decoder layers 704. In some embodiments, the small LLM model 700 has the same number of decoder layers as encoder layers. In an example embodiment, the small LLM model 700 has three decoder layers.

The head 705 is a head that outputs a prediction of which terms included in masked patient notes were masked.

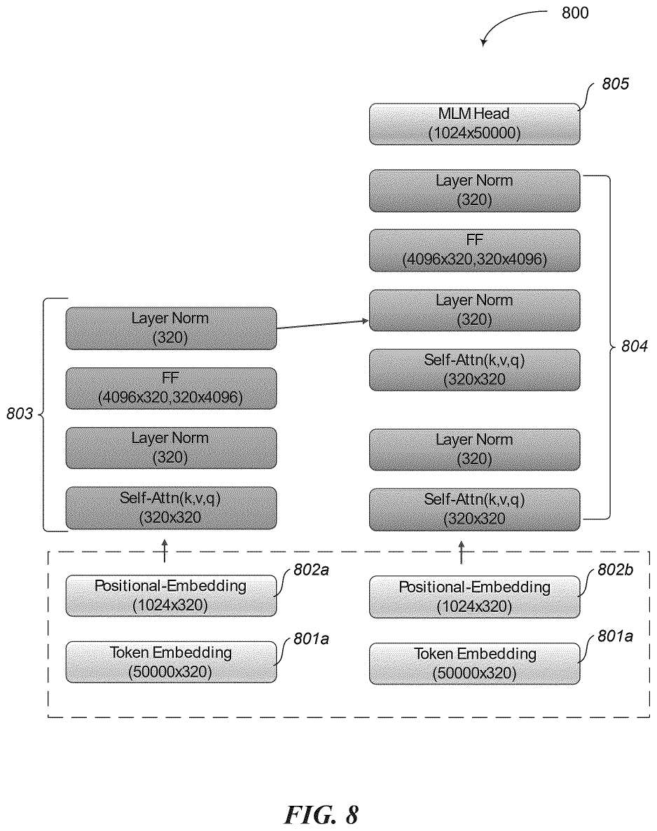

FIG. 8 is a block diagram of a sample small LLM model 800 after embeddings are frozen, used by the facility in some embodiments. The small LLM model 800 includes one or more token embedding blocks 801a and 801b (collectively as “token embedding blocks 801” or individually as “token embedding block 801”), one or more positional embedding blocks 802a and 802b (collectively as “positional embedding blocks 802” or individually as “positional embedding block 802”), one or more encoder layers 803, one or more decoder layers 804, and a head 805. The token embedding blocks 801, positional embedding blocks 802, encoder layer 803, decoder layer 804, and head 805 may be similar to the token embedding blocks 701, positional embedding blocks 702, encoder layer 703, and decoder layer 704, respectively, described above in connection with FIG. 7.

The head 805 may be different head from the head 705, described above in connection with FIG. 7. In an example embodiment, the head 805 is a head that outputs a dictionary including medical information extracted from one or more patient notes. In some embodiments, the head 805 is a head layer that outputs a determination of whether one or more patient notes indicate a specified medical condition. In some embodiments, the head 805 is a head layer that outputs a determination of whether one or more patient notes indicate a serious health condition. In some embodiments, the head 805 is a head layer that outputs an answer to a specified health question.

The token embedding blocks 801 and positional embedding blocks 802 of the small LLM model 800 are frozen, such that subsequent training of the small LLM model 800 does not change the token embedding blocks 801 and positional embedding blocks 802. Thus, the facility may train the small LLM model 800 without changing the model's 800 interpretation of tokens generated by a tokenizer, such as the tokenizer generated by using the process 200 described above in connection with FIG. 2.

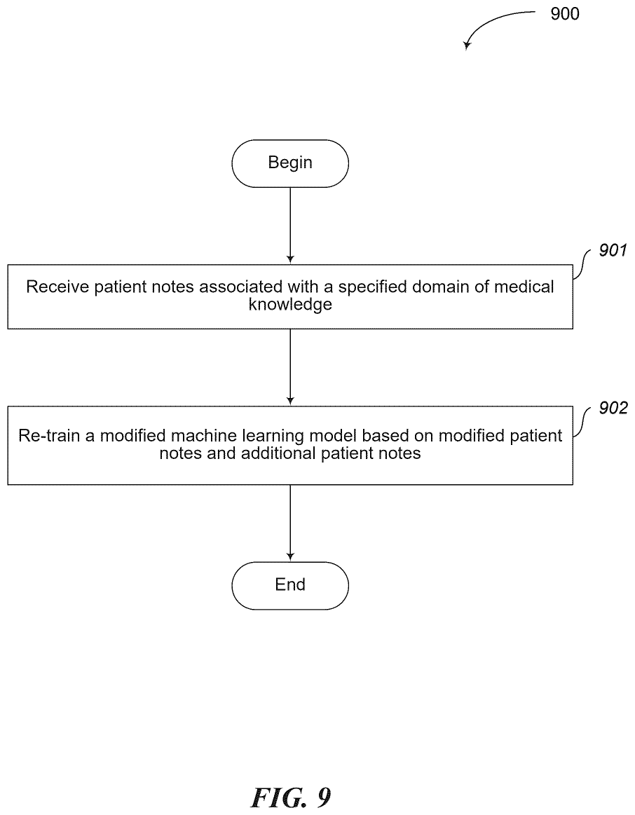

FIG. 9 is a flow diagram showing a process 900 to re-train a small LLM model, performed by the facility in some embodiments. First, at act 901, the facility receives patient notes associated with a specified domain of medical knowledge. In some embodiments, the facility receives the patient notes associated with a specified domain of medical knowledge in a similar manner to act 201, described above in connection with FIG. 2. In some embodiments, the facility generates synthetic patient notes based on the patient notes received in act 901 by using the process 300 described above in connection with FIG. 3. In some embodiments, the facility modifies at least a portion of the received patient notes to include noise, in a similar manner to act 502, described above in connection with FIG. 5. In some embodiments, the facility modifies at least a portion of the received patient notes to include synonyms for at least one word included in the patient notes, in a similar manner to act 503, described above in connection with FIG. 5.

At act 902, the facility re-trains a modified machine learning model based on modified patient notes and additional patient notes. In some embodiments, the re-trained machine learning model is a machine learning model trained based on the process 600, described above in connection with FIG. 6. In some embodiments, the modified patient notes are modified patient notes used to originally train the machine learning model.

After act 902, the process 900 ends.

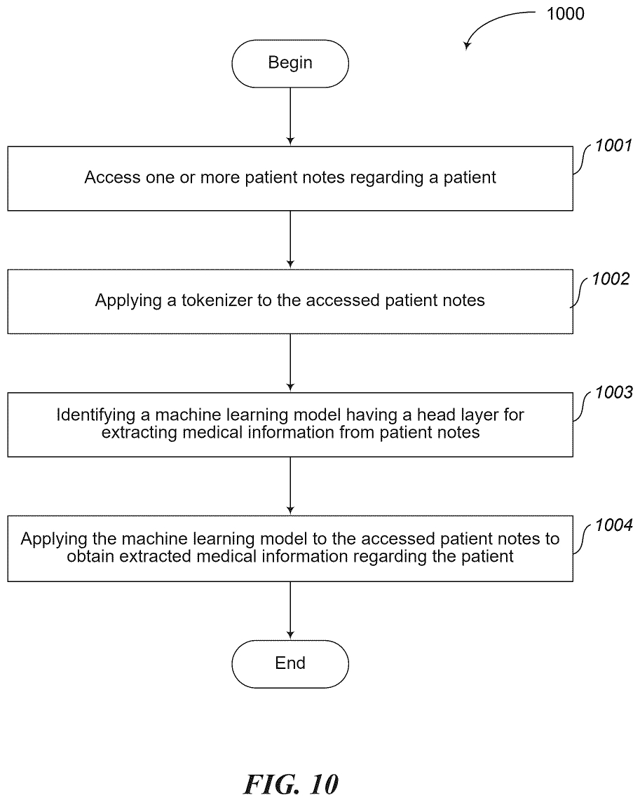

FIG. 10 is a flow diagram showing a process 1000 for using a small LLM model, performed by the facility in some embodiments. First, at act 1001, the facility accesses one or more patient notes regarding a specified patient.

At act 1002, the facility applies a tokenizer to the accessed patient notes. In some embodiments, the tokenizer is a tokenizer trained by using the process 200, described above in connection with FIG. 2.

At act 1003, the facility identifies a machine learning model having a head layer for extracting medical information from patient notes. In some embodiments, the machine learning model is a small LLM model, such as the small LLM model trained by the process 600 described above in connection with FIG. 6, or the small LLM model 800 described above in connection with FIG. 8.

At act 1004, the facility applies the machine learning model to the accessed patient notes to obtain extracted medical information regarding the patient.

After act 1004, the process 1000 ends.

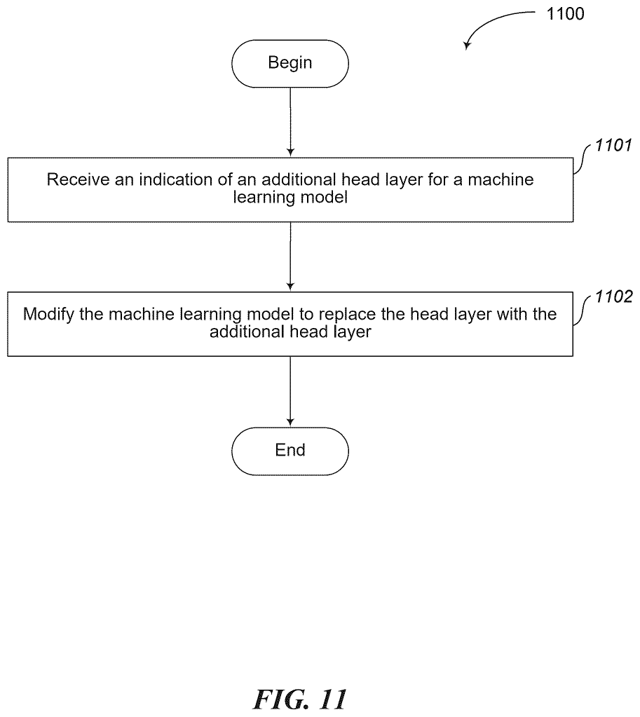

FIG. 11 is a flow diagram showing a process 1100 for changing the head of a small LLM model, performed by the facility in some embodiments. First, at act 1101, the facility receives an indication of an additional head layer for a machine learning model. In some embodiments, the additional head layer is a head layer that outputs a determination of whether one or more patient notes indicate a specified medical condition. In some embodiments, the additional head layer is a head layer that outputs a determination of whether one or more patient notes indicate a serious health condition. In some embodiments, the additional head layer is a head layer that outputs an answer to a specified health question. In some embodiments, the additional head layer is a conditional generation head layer that outputs a dictionary that includes medical information extracted from one or more patient notes.

At act 1102, the facility modifies the machine learning model to replace the head layer with the additional head layer. In some embodiments, as part of modifying the machine learning model to replace the head layer with the additional head layer, the facility modifies the machine learning model to receive additional input. For example, if the additional head layer outputs an answer to a specified health question, the facility may modify the machine learning model to receive input indicating the specified health question.

After act 1102, the process 1100 ends.

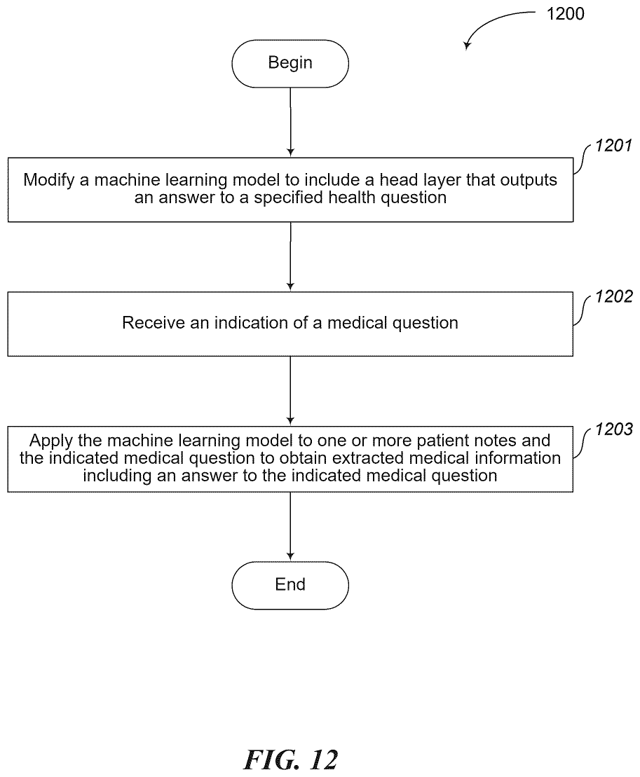

FIG. 12 is a flow diagram showing a process 1200 for using a small LLM model with a medical question extraction head, performed by the facility in some embodiments. First, at act 1201, the facility modifies a machine learning model to include a head layer that outputs an answer to a specified health question. In some embodiments, the machine learning model is a small LLM model, such as the small LLM model 800 described above in connection with FIG. 8.

At act 1202, the facility receives an indication of a medical question. In some embodiments, the facility receives the indication of the medical question via user input.

At act 1203, the facility applies the machine learning model to one or more patient notes and the indicated medical question to obtain extracted medical information including an answer to the indicated medical question. In some embodiments, the facility performs act 1203 in a similar manner to act 1001, described above in connection with FIG. 10.

After act 1203, the process 1200 ends.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system, comprising:
receiving an indication of one or more patient notes;
for each patient note of the one or more patient notes:
masking at least a portion of the patient note;
training a machine learning model based on the masked patient notes, the machine learning model having one or more embedding layers and a first head layer that determines an output of the machine learning model;
freezing at least one embedding layer of the machine learning model;
generating modified patient notes by:
for at least one of the one or more patient notes, for each word of one or more words in the patient note, modifying the word to be a synonym of the word;
modifying the machine learning model to replace the first head layer of the machine learning model with a second head layer of the machine learning model; and
training the modified machine learning model based on the modified patient notes, such that the machine learning extracts medical information from patient notes.

2. The method of claim 1, wherein a portion of the words modified to be a synonym are medical terms.

3. The method of claim 1, wherein the second head layer is a conditional generation head layer.

4. The method of claim 1, wherein training the machine learning model further comprises:
receiving an indication of one or more synthetic patient notes; and
training the modified machine learning model based on the modified patient notes and the one or more synthetic patient notes.

5. The method of claim 4, receiving the indication of one or more synthetic patient notes further comprises:
generating a prompt for a machine learning model trained to generate text based on a prompt; and
generating synthetic patient notes by applying the prompt to the machine learning model.

6. The method of claim 4, receiving the indication of one or more synthetic patient notes further comprises:
accessing a repository of patient notes; and
generating synthetic patient notes by selecting one or more patient notes from the repository of patient notes.

7. The method of claim 1, wherein masking at least a portion of the patient note further comprises:
randomly selecting one or more words included in the patient note; and
masking the randomly selected one or more words.

8. The method of claim 1, wherein the one or more patient notes are directed to a first domain of medical knowledge and the method further comprises:
receiving an indication of additional patient notes associated with a second domain of medical knowledge; and
re-training the modified machine learning model based on the modified patient notes and additional patient notes.

9. The method of claim 8, wherein the machine learning model is a Bart machine learning model that comprises:
at least one encoder layer; and
at least one decoder layer.

10. One or more instances of computer-readable media not constituting a transitory propagating data signal, the one or more instances of computer-readable media collectively having contents configured to cause a computing device to perform a method comprising:
receiving an indication of one or more patient notes;
for each patient note of the one or more patient notes:
masking at least a portion of the patient note;
training a machine learning model based on the masked patient notes, the machine learning model having one or more embedding layers and a first head layer that determines an output of the machine learning model;
freezing at least one embedding layer of the machine learning model;
generating modified patient notes by:
for at least one of the one or more patient notes, for each word of one or more words in the patient note, modifying the word to be a synonym of the word;
modifying the machine learning model to replace the first head layer of the machine learning model with a second head layer of the machine learning model; and
training the modified machine learning model based on the modified patient notes, such that the machine learning model outputs a dictionary.

11. A method in a computing system, comprising:
receiving an indication of patient notes, at least a portion of the patient notes being artificially generated to train a tokenizer; and
training the tokenizer based on the patient notes.

12. The method of claim 11, further comprising:
applying the output of the trained tokenizer to a machine learning model trained to extract information from patient notes.

13. The method of claim 11, wherein receiving the indication of patient notes comprises:
generating a prompt for a machine learning model trained to generate text based on a prompt; and
generating synthetic patient notes by applying the prompt to the machine learning model.

14. The method of claim 13, wherein generating the prompt comprises:
select one or more sample dictionaries, each dictionary including an indication of at least one data class and at least one data type associated with each data class; and
generate the prompt for the machine learning model based on the one or more sample dictionaries.

15. The method of claim 14, further comprising:
validating the synthetic patient notes based on the indicated at least one data class and indicated at least one data type included in each of the one or more sample dictionaries; and
based on the validation of the synthetic patient notes, determining whether the synthetic patient notes is to be used to train the tokenizer.

16. The method of claim 11, wherein receiving the indication of patient notes comprises:
modifying at least a portion of the indicated patient notes to include noise.

17. The method of claim 11, wherein receiving the indication of patient notes comprises:
for each patient note of at least a portion of the indicated patient notes:
modifying at least one word in the patient note to be a synonym of the at least one word.

18. A method in a computing system, comprising:
receiving an indication of one or more patient notes regarding a patient;
identifying a machine learning model, the machine learning model having a head layer for extracting medical information from patient notes; and
applying the machine learning model to the one or more patient notes to obtain extracted medical information regarding the patient.

19. The method of claim 18, further comprising:
receiving an indication of an additional head layer for the machine learning model;
modifying the machine learning model to replace the head layer with the additional head layer.

20. The method of claim 19, wherein receiving the indication of the additional head layer further comprises:
receiving an indication of a type of medical information to be extracted; and
selecting a head layer of a plurality of head layers based on the type of medical information to be extracted.

21. The method of claim 18, wherein the head layer of the machine learning model causes the machine learning model to output a dictionary that includes extracted medical information.

22. The method of claim 18, wherein the machine learning model is compatible with a plurality of head layers, comprising:
a head layer that outputs a dictionary that includes medical information extracted from patient notes;
a head layer that outputs a determination of whether patient notes indicate a specified medical condition;
a head layer that outputs a determination of whether patient notes indicate a serious health condition; or
a head layer that outputs an answer to a specified health question.

23. The method of claim 18, wherein the head layer of the machine learning model outputs an answer to a specified health question and the method further comprises:
receiving an indication of medical question; and
applying the machine learning model to the one or more patient notes and the indicated medical question to obtain extracted medical information regarding a patient, the extracted medical information including an answer to the indicated medical question.
